(19) United States
(12) Patent Application Publication
(10) Pub. No.: US 2025/0061998 A1
(43) Pub. Date: Feb. 20, 2025

(54) SYSTEM AND METHOD FOR TREATMENT OF CHRONIC LOW BACK PAIN WITH DIGITAL THERAPEUTICS

(71) Applicant: JOGO HEALTH, INC., BRIDGEWATER, NJ (US)

(72) Inventors: Gary KRASILOVSKY, Bridgewater, NJ (US); Michelle DELA CRUZ, Bridgewater, NJ (US)

(73) Assignee: JOGO HEALTH, INC., BRIDGEWATER, NJ (US)

(21) Appl. No.: 18/723,925

(22) PCT Filed: Dec. 28, 2022
(86) PCT No.: PCT/US2022/054154

§ 371 Date: Jun. 25, 2024

Related U.S. Application Data
(60) Provisional application No. 63/294,720, filed on Dec. 29, 2021.

(51) Int. Cl. G16H 20/30; G16H 10/20; G16H 80/00

(52) U.S. Cl. CPC G16H 20/30; G16H 10/20; G16H 80/00

(57) ABSTRACT

A system and method for remote assessment by telemedicine of a patient with chronic low back pain are described. The system includes an electromyography sensor worn by the patient and used as part of a biofeedback therapy. The patient is assessed based on a plurality of subjective questionnaires and on an objective assessment performed by a physical therapist or other clinician by telemedicine. The treatment protocols are designed so that the patient can perform them at home with remote monitoring by a physical therapist.

### BACKGROUND

Low back pain (LBP) is one of the most common ailments to affect adults and is one of the most common reasons for a patient to seek physical therapy treatment (PT). Despite recent progress in healthcare, the associated global prevalence and economic strain of this condition continues to grow, especially among adults in industrialized countries, wherein LBP is the most widespread reason for lost wages and disability. Of this group, about 10-30% of these cases become chronic low back pain (CLBP) resulting in further socioeconomic burden such as lost time and money on failed treatments, sick leave, and overall suffering.

For over fifty years, biofeedback has been utilized in rehabilitation to normalize post-injury related movement patterns like gait. Biofeedback relays real-time biological information/measurements to patients that would otherwise be intangible to them. Biofeedback employs the use of various equipment, instruments, or procedures in measuring different types of biological data such as cardiovascular, neuromuscular, or even respiratory data. This real-time data provided to the patient can be in the form of audio, visual, or numerical displays and is then mentally processed by the patient to promote increased awareness and self-regulation over the physiological process is being measured.

The presence of higher muscle activation at baseline and irregular electromyography (EMG) patterns in those with a history of LBP has been suggested. Those with LBP are believed to demonstrate higher EMG especially in standing versus those without. In addition, there is a deficit or absence in relaxation patterns during maximum flexion of lumbar musculature, a phenomenon present in CLBP patients, which have been associated with pain, disability, and fear avoidance.

### DESCRIPTION OF THE DRAWINGS

FIG. 1 is an example system diagram.

FIG. 2 is an example flow diagram of a method for assessing a patient and assigning treatment protocols.

FIG. 3 is an example computer system.

### DETAILED DESCRIPTION

Those skilled in the art will recognize other detailed designs and methods that can be developed employing the teachings of the present invention. The examples provided here are illustrative and do not limit the scope of the invention, which is defined by the attached claims. The following detailed description refers to the accompanying drawings. The same reference numbers in different drawings may identify the same or similar elements.

Consistent with implementations described herein, systems and methods are described that use biofeedback in treatment of CLBP, which can lead to short and long-term improvements in pain-related outcomes such as pain intensity, muscle tension, disability, and depression, and can be beneficial as either an isolated treatment or used in conjunction with other interventions.

In an example method, physical therapy, pain neuroscience education, and surface electromyographic based biofeedback (sEMG-BF) are used to accurately assess the underlying causes of neuromuscular conditions including pain, and provide means for intervention. The systems and methods herein may use principles of neuroplasticity and biofeedback to empower a patient to be able to demonstrate self-regulation, specifically strengthening their brain's control over their own muscles in producing or inhibiting movement. Over time, the benefits experienced during this training can have lasting carryover effects beyond the use of the systems and methods described herein due to neuroplasticity, which is the formation of neural pathways responsible for a specific movement related goal.

In an aspect of the invention, an evidence driven protocol for CLBP is used in conjunction with a portable and wearable surface EMG sensor device paired with digital biofeedback software application pre-installed on a tablet computer. This protocol is designed to be implemented via telemedicine via a kit sent to the patient's home for usage as advised by clinicians. This protocol includes both a subjective and objective assessment to filter each patient into an appropriate treatment and intervention group.

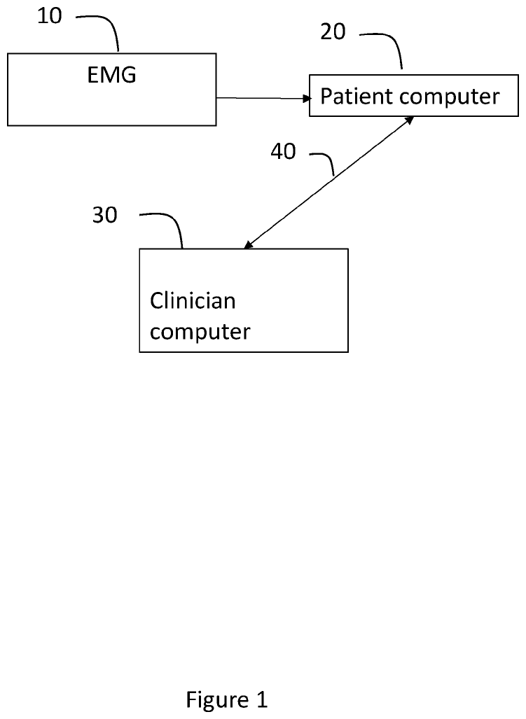

FIG. 1 is a system block diagram of an example system for performing the methods described herein. The system includes a patient wearable EMG 10, a patient network connection and computing device 20, a clinician computing device 30 and a wide area network 40. The patient network connection and computing device 20 is connected to the EMG to provide EMG feedback to the patient and EMG data to the clinician. The patient network connection and computing device may be a desktop computer, a laptop computer, a tablet, a smart phone, a dedicated processor-based device specifically designed for use by the patient in performing the methods described herein or any other suitable processing device capable of receiving EMG data, communicating over a wide area network and displaying information to the patient. The clinician computing device may be desktop computer, a laptop computer, a tablet, a smart phone or a dedicated processor-based device specifically designed for use by the clinician in performing the methods described herein.

Table 1 is a breakdown of subjective and objective assessments used to allocate patients to one of three treatment groups. Group 1 patients will use physical therapy, pain neuroscience education (PNE, explained below) and EMG biofeedback. Group 2 patients will use progressive muscle relaxation (PMR), PNE and EMG biofeedback. Group 3 patients will use physical therapy and EMG biofeedback. The legend for Table 1 is as follows: +ODI indicates a score of greater than sixty on the Owestry Disability Index. +FABQ indicates a score of greater than twenty on the Fear Avoidance Questionnaire. +CSI indicates a score of greater than fifty on the Central Sensitization Inventory. +PSI indicates a score of greater than five on the Pittsburgh Sleep Quality Index. OMA stands for Objective Movement Assessment. Not all of the outcomes are likely to occur from a practical standpoint. The most likely outcomes are numbered 1-16, 19, 21, 27 and 29 in the left-hand column of Table 1. The applicable treatment protocol group (Tx) is shown in the right-hand column.

TABLE 1

| | | | | | | TX |
|---|---|---|---|---|---|---|
| | ODI | FABQ | CSI | PSI | OMA | GROUP |
| 1 | + | + | + | + | + | 1 |
| 2 | + | + | + | − | + | 1 |
| 3 | + | + | − | − | + | 1 |
| 4 | + | + | − | + | + | 1 |
| 5 | + | − | − | − | + | 1 |
| 6 | + | − | − | + | + | 1 |
| 7 | + | − | + | + | + | 1 |
| 8 | + | − | + | − | + | 1 |
| 9 | + | + | + | + | − | 2 |
| 10 | + | + | + | − | − | 2 |
| 11 | + | + | − | − | − | 2 |
| 12 | + | + | − | + | − | 2 |
| 13 | + | − | − | − | − | 2 |
| 14 | + | − | − | + | − | 2 |
| 15 | + | − | + | + | − | 2 |
| 16 | + | − | + | − | − | 2 |
| 17 | − | + | + | + | + | 3 |
| 18 | − | + | + | − | + | 3 |
| 19 | − | + | − | − | + | 3 |
| 20 | − | + | − | + | + | 3 |
| 21 | − | − | − | − | + | 3 |
| 22 | − | − | − | + | + | 3 |
| 23 | − | − | + | + | + | 3 |
| 24 | − | − | + | − | + | 3 |
| 25 | − | + | + | + | − | 2 |
| 26 | − | + | + | − | − | 2 |
| 27 | − | + | − | − | − | 2 |
| 28 | − | + | − | + | − | 2 |
| 29 | − | − | − | − | − | 2 |
| 30 | − | − | − | + | − | 2 |
| 31 | − | − | + | + | − | 2 |
| 32 | − | − | + | − | − | 2 |

### Subjective Outcome Measures

Patients will complete questionnaires based on validated and published subjective tests and then the results will be analyzed to determine the appropriate intervention and treatment group for each patient. The Oswestry Disability Index (ODI) is a questionnaire measuring a patient's perceived functional disability as it relates to back pain in various activities of daily living (ADL). The outcome measures evaluate chronic pain-related factors such as aspects of the patient's perceived functional disability, central sensitization, fear avoidance behaviors, and sleep quality. In addition to the ODI, patients will be given a fear avoidance behavior questionnaire (FABQ), a central sensitization inventory (CSI) and a questionnaire to assess sleep quality based on the Pittsburgh Sleep Quality Index (PSI).

### Objective Examination

An objective movement assessment (OMA) is used for the practitioner (e.g. a physical therapist) to objectively assess, via telemedicine, the quality of a patient's gross spinal and complex functional movements that may have an impact on chronic low back pain. The OMA helps to identify compensatory movement strategies and faulty movement patterns that may have developed due to pain or as a result of pain. The OMA may be performed in conjunction with reception by the practitioner of patient EMG data.

### Group 1 Treatment Protocol

Patients that fall into this group have scored above threshold (+) on the ODI and have scored above threshold (+) in at least one of the three subjective measures (FABQ, CSI, or PSI), and were also able to complete the OMA.

Patients in this group are given (PNE), which is Pain Neuroscience Education, and includes physical therapist-provided patient education as a treatment where deemed applicable. Patient education is given primary importance when CSI, FAB-Q, and/or PSI are significant and exceeds thresholds (+) as it precedes the immediate need for traditional biomechanical focus, as in customary PT treatments. After a retest period, EMG biofeedback will be used to strengthen weak or inhibit hypertonic musculature. EMG-biofeedback will be used to assess and train recruitment of specific muscles based on the assessment, as well as teach mindful movement or resting postures.

### Group 2 Treatment Protocol

Patients that fall into this group have scored above threshold (+) on the ODI and have scored above threshold (+) in at least one of the three subjective measures (FABQ, CSI, or PSI), but were not able to complete the OMA as demonstrated by inability to perform, complete, or complain of fear avoidance, pain, or tightness, with multiple movements. The second group that may fall into this treatment category have scored below threshold on the ODI and other questionnaires except the FABQ and were not able to complete the OMA.

Patients in this group are treated with progressive muscle relaxation (PMR), PNE and EMG biofeedback. With PMR, muscle groups are tensed or contracted then released across various areas of the body. This technique has been modified or offered inspiration for other mind-body treatment methods for multiple stress-involving ailments like headaches, insomnia, and chronic pain. EMG biofeedback will be used in conjunction with principles of PMR to assess overactive muscles and train patients to relax or “down train” their hypertonic or overactive muscle groups. Sites of relaxation may be targeted as guided by the patient's subjective history or complaints, along with patient's postures, or performance or lack thereof on the OMA. Surface EMG (sEMG) readings of elevated tension in a muscle or lack of recovery of the muscle following an increase in tension, will include PMR and down training in the intervention. sEMG readings are recorded from the same muscle on both sides of the patient to determine evidence of asymmetry. PNE may be offered to this group as determined by a physical therapist.

### Group 3 Treatment Protocol

Patients that fall into this group had below threshold scores (−) on the subjective assessments described above and were able to complete the OMA, demonstrating identifiable areas for customary PT treatment of biomechanical focus but strengthened by the use of EMG biofeedback. The second group that falls into this treatment category have scored below threshold (−) on the ODI, have potentially scored above threshold on FABQ, but were still able to complete the OMA. Physical therapy for this group includes movement re-education, mobility and strengthening exercises and postural correction. In this group, EMG biofeedback will be used to assess and train recruitment of specific muscles based on the assessment and the patient will be prescribed a home program for independent use of a PT-prescribed EMG biofeedback protocol, as follows:

The PT will determine movement from the Objective Movement Assessment (OMA) that was the most impaired, painful, or dysfunctional. The PT will then use an assessment that targets muscle in the lumbo-pelvic region that may be weak, having a timing and coordination issue, or a motor control issue. There are five myofascial lines that provide support to the lumbopelvic region: the superficial front line, the superficial back line, the deep front line, the lateral line and the spiral line. An exemplary assessment targets the main muscle groups in each of these lines. The therapist cues the patient to activate a target muscle from each of the five lines and once the patient accurately activates the muscle the therapist asks the patient to perform the dysfunctional movement found in the OMA. The therapist then determines the myofascial line that provided the most improvement in the categories of pain, range of motion, and motor control. Once the line is determined by the therapist, the therapist can begin using EMG biofeedback on the appropriate muscle or muscle group for treatment.

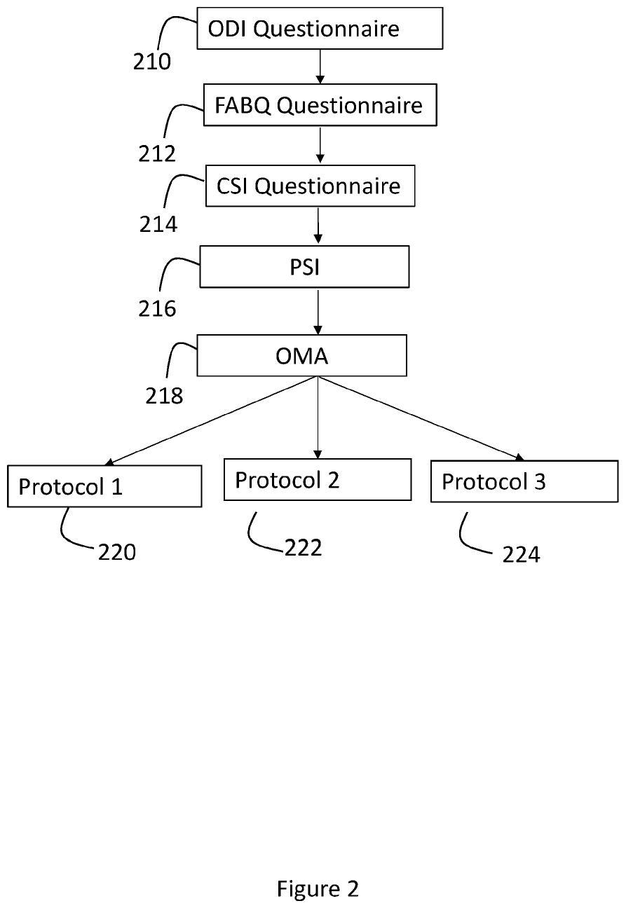

FIG. 2 is a flow diagram describing an example process for assessing a patient and determining a treatment protocol based on the assessment. At step 210, a patient completes an Oswestry Disability Index questionnaire regarding a patient's perceived functional disability as it relates to back pain in various activities of daily living. At step 212, the patient completes a fear avoidance behavior questionnaire (FABQ). At step 214, the patient completes a central sensitization inventory (CSI) questionnaire and at step 216 the patient completes a questionnaire to assess sleep quality based on the Pittsburgh Sleep Quality Index (PSI). At step 218, an objective movement assessment (OMA) is made by a clinician (e.g. a physical therapist) to objectively assess, via telemedicine, the quality of a patient's gross spinal and complex functional movements that may have an impact on chronic low back pain. Based on the outcome of steps 210, 212, 214, 216 and 218, the patient is assigned to one of three treatment protocols, as described in detail above. The details of the assignment process are listed in Table 1.

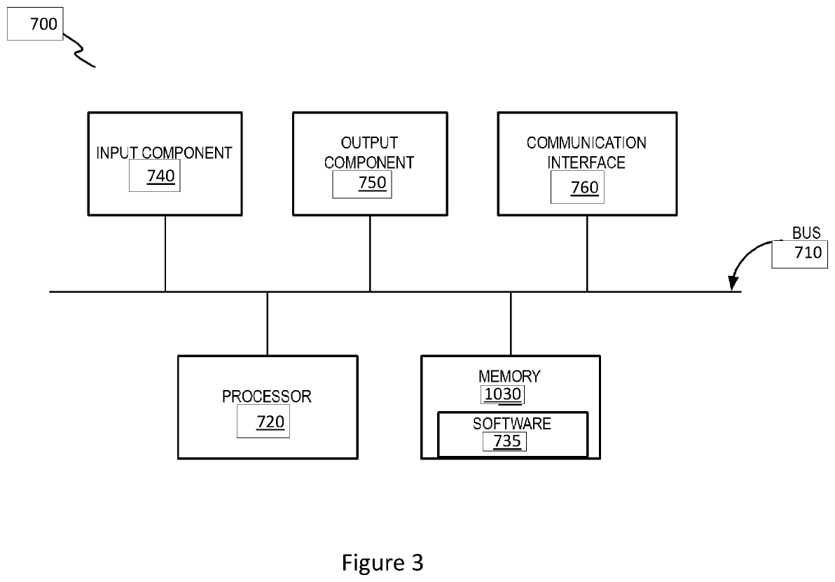

FIG. 3 is a diagram illustrating exemplary physical components of a device 700. Device 700 may correspond to various devices within the above-described system, such as a computer, tablet or smart phone used at home by the patient and may be connected to receive EMG data from the patient. Device 700 may also be a computer, tablet or smartphone, for example that is used by a clinician to observe a patient and/or the patient's EMG data via a network. Device 700 may include a bus 710, a processor 720, a memory 730, an input component 740, an output component 750, and a communication interface 760.

Bus 710 may include a path that permits communication among the components of device 700. Processor 720 may include a processor, a microprocessor, or processing logic that may interpret and execute instructions. Memory 730 may include any type of dynamic storage device that may store information and instructions, for execution by processor 720, and/or any type of non-volatile storage device that may store information for use by processor 720.

Software 735 includes an application or a program that provides a function and/or a process. Software 735 is also intended to include firmware, middleware, microcode, hardware description language (HDL), and/or other form of instruction. By way of example, with respect to the network elements that include logic to provide proof of work authentication, these network elements may be implemented to include software 735. Additionally, for example, device 700 may include software 735 to perform tasks as described above with respect to FIGS. 2-6.

Input component 740 may include a mechanism that permits a user to input information to device 700, such as a keyboard, a keypad, a button, a switch, etc. Output component 750 may include a mechanism that outputs information to the user, such as a display, a speaker, one or more light emitting diodes (LEDs), etc.

Communication interface 760 may include a transceiver that enables device 700 to communicate with other devices and/or systems via wireless communications, wired communications, or a combination of wireless and wired communications. For example, communication interface 760 may include mechanisms for communicating with another device or system via a network. Communication interface 760 may include an antenna assembly for transmission and/or reception of RF signals. In one implementation, for example, communication interface 760 may communicate with a network and/or devices connected to a network. Alternatively or additionally, communication interface 760 may be a logical component that includes input and output ports, input and output systems, and/or other input and output components that facilitate the transmission of data to other devices.

Device 700 may perform certain operations in response to processor 720 executing software instructions (e.g., software 735) contained in a computer-readable medium, such as memory 730. A computer-readable medium may be defined as a non-transitory memory device. A non-transitory memory device may include memory space within a single physical memory device or spread across multiple physical memory devices. The software instructions may be read into memory 730 from another computer-readable medium or from another device. The software instructions contained in memory 730 may cause processor 720 to perform processes described herein. Alternatively, hardwired circuitry may be used in place of or in combination with software instructions to implement processes described herein. Thus, implementations described herein are not limited to any specific combination of hardware circuitry and software.

Device 700 may include fewer components, additional components, different components, and/or differently arranged components than those illustrated in FIG. 7. As an example, in some implementations, a display may not be included in device 700. In these situations, device 700 may be a “headless” device that does not include input component 740. Additionally, or alternatively, one or more components of device 700 may perform one or more tasks described as being performed by one or more other components of device 700.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

What is claimed is:

1. A method for assigning a patient with chronic low back pain to one of a plurality of treatment protocols, the method comprising the steps of:
evaluating the patient based on the Owestry Disability Index (ODI),
evaluating the patient based on a Fear Avoidance Behavior Questionnaire (FABQ),
evaluating the patient based on a Central Sensitization Inventory (CSI),
evaluating the patient based on the Pittsburgh Sleep Quality Index (PSI), and
evaluating the patient based on an objective movement assessment (OMA) using an EMG sensor on the patent, and
assigning the patient to a first treatment protocol in the case where the patient has scored above a threshold on the ODI, has scored above predetermined thresholds on at least one of the FABQ, CSI and PSI, and where the patient has successfully completed the OMA.

2. The method of claim 1, wherein the evaluating of the patient is performed over a wide area network by a clinician remote from the patient.

3. The method of claim 1, wherein the OMA comprises a full body movement screen assessing spinal range of motion, quality of movement, and painful movements.

4. The method of claim 1, further comprising assigning the patient to a second treatment protocol in the case where the patient has scored above the threshold on the ODI, has scored above the predetermined thresholds on at least one of the FABQ, CSI and PSI, and where the patient has not successfully completed the OMA.

5. The method of claim 1, further comprising assigning the patient to a third treatment protocol in the case where the patient has scored below the threshold on the ODI, has scored below the predetermined thresholds on all of the FABQ, CSI and PSI, and where the patient has successfully completed the OMA.

6. The method of claim 1, wherein the first treatment protocol includes: performing physical therapy, applying pain neuroscience education and applying biofeedback using an EMG.

7. The method of claim 4, wherein the second treatment protocol includes: performing progressive muscle relaxation, applying pain neuroscience education and applying biofeedback using an EMG.

8. The method of claim 5, wherein the third treatment protocol includes: performing physical therapy and applying biofeedback using an EMG.
